(19) United States
(12) Patent Application Publication
(10) Pub. No.: US 2018/0036332 A1
(43) Pub. Date: Feb. 8, 2018

(54) USE OF NADPH IN PREPARING MEDICINES FOR TREATMENT OF HEART DISEASES

(71) Applicant: Chongqing Institute of Bench-to Bed Bioengineering Technology Co., Ltd., Chongqing (CN)

(72) Inventors: Zheng-hong QIN, Suzhou (CN); Mei LI, Suzhou (CN)

(73) Assignee: Chongqing Institute of Bench-to Bed Bioengineering Technology Co., Ltd., Chongqing (CN)

(21) Appl. No.: 15/551,367

(22) PCT Filed: Nov. 24, 2015
(86) PCT No.: PCT/CN2015/095391

§ 371 Date: Aug. 16, 2017

(30) Foreign Application Priority Data
Feb. 17, 2015 (CN) 201510086548.7

(51) Int. Cl. A61K 31/7084

(52) U.S. Cl. CPC A61K 31/7084

(57) ABSTRACT

The use of reduced nicotinamide adenine dinucleotide phosphate (NADPH) in preparing medicines for the treatment of heart diseases, and medicines for the treatment of heart diseases using NADPH as the active ingredient, said heart diseases including cardiac damage, myocardial infarction, and cardiomyopathy.

### TECHNICAL FIELD

The present invention relates to a new use of NADPH, in particular to a use of NADPH in preparing medicines for treatment of heart diseases.

### BACKGROUND OF THE INVENTION

Cardiovascular and cerebrovascular diseases refer to diseases of heart blood vessels and diseases of cerebral blood vessels. Cardiovascular and cerebrovascular diseases have the features of high morbidity rate, high mortality rate, high disability rate, high recurrence rate, and numerous complications (four “high” and one “numerous”). It has exerted a heavy burden on the global health care and medical resources now, and is the top enemy of the second medical revolution. Currently, there are over 270 million patients suffering from cardiovascular and cerebrovascular diseases in China. Particularly as the society is becoming an aging population society, the morbidity of cardiovascular and cerebrovascular diseases is increasing continuously. Therefore, investigating the pathological mechanism, prevention and treatment of cardiovascular and cerebrovascular diseases are important tasks in the medical field. The pathogenic mechanism of cardio-cerebral ischemia disease is very complicated. Investigating the pathogenic mechanism of cardio-cerebral ischemia disease and identify novel target for medicine are of great practical significance for developing medicines for preventing and treating cardiovascular and cerebrovascular diseases. In cardiovascular and cerebrovascular diseases, as the heart bears the role of providing power in the circulation system, the pathogenesis of heart has its particularity compared to that of other organs.

Nicotinamide adenine dinucleotide phosphate (NADPH) is produced by metabolism of glucose through a pentose phosphate pathway (PPP). It is the most important electron donor in cells and a reducing agent for biosynthesis that provides hydrogen ions for reduction type biosynthesis. NADPH is a coenzyme of glutathione (GSH) reductase and can convert oxidized glutathione (GSSG) to the reduced GSH, through which a normal content of the reduced GSH is maintained. GSH is an important antioxidant in cells that can protect certain sulfhydryl containing proteins, lipid and protease from being damaged by oxidants. It has especially important role in maintaining erythrocyte membrane. NADPH not only participates in the biosynthesis of cholesterol, fatty acids, monooxygenase species, steroid hormones, but also participates in the hydroxylation reaction in vivo and biological conversion of drugs, toxicants and certain hormones. For example, NADPH can utilize the electron donor from a detoxification cell to maintain the balance of oxidation and reduction by reducing oxidation type compounds through metabolism in vivo, which plays an important role in the oxidation defense system. NADPH can also enter the respiratory chain to generate ATP through the shuttle action of the isocitric acid: due to the low permeability of the mitochondrial inner membrane to substances, the NADPH generated outside of the mitochondria cannot directly enter the respiratory chain to be oxidized. The H on NADPH can be delivered to NAD+ under the action of isocitric acid dehydrogenase, and then enters the respiration chain through NAD+ to generate energy. The maintenance of cell energy metabolism and reduction of ROS (reactive oxygen species) are critical to cell survival and especially to the survival of tissues with ischemia and anoxia. It is commonly accepted that energy metabolism disorder and oxidative stress are important mechanisms of the cardio-cerebral ischemia disease. Researches have showed that increasing cell energy metabolism capability and reducing cell ROS generation can ameliorate cell damage induced by ischemia and anoxia. Based on the multiple physiological functions of NADPH, some studies have been carried out to apply NADPH to the treatment of certain diseases.

According to the report of an international literature “p53 and TIGAR regulate cardiac myocyte energy homeostasis under hypoxic stress”, knocking off TIGAR or upstream regulatory genes of TIGAR from the myocardial cells has an effect of increasing the apoptosis of myocardial cells under hypoxic stress. TIGAR inhibits the glycolysis pathway and activates the pentose bypass pathway which generates two metabolites: −NADPH and 5-phosphopentose. Therefore, knocking off TIGAR enhances the glycolysis and decreases the activity of the pentose metabolism, wherein the inhibition of pentose pathway means less NADPH is produced, which suggests that NAPDH exacerbates the myocardial injury. Thus, there has been no study so far to use NAPDH in the treatment of heart diseases.

### SUMMARY OF THE INVENTION

The present invention aims to provide a new use of NAPDH, namely the use of NADPH in preparation of a medicine for treating a heart disease.

The present invention provides use of NADPH in preparation of a medicine for treating a heart disease.

Further, the heart disease is selected from the group of myocardial injury, myocardial infarction and cardiomyopathy.

Further, the cardiomyopathy is a hypertrophic cardiomyopathy.

The medicine comprises a pharmaceutically effective amount of NADPH and a pharmaceutically acceptable carrier.

The present invention provides a medicine for treating heart diseases, wherein NADPH is the active ingredient of the medicine, and the medicine is prepared by adding conventional auxiliary ingredients to NADPH so as to produce a clinically acceptable mixture, capsule, tablet, medicinal film agent or spraying agent according to a conventional process.

The present invention provides a medicine for treating myocardial injury, myocardial infarction or cardiomyopathy, wherein NADPH is the active ingredient of the medicine, and the medicine is prepared by adding conventional auxiliary ingredients to NADPH to produce a clinically acceptable mixture, capsule, tablet, medicinal film agent or spraying agent according to a conventional process.

According to the technical scheme of the present invention, NADPH is used as the active ingredient for preparing medicines for treating a heart disease. It is proved that NADPH has the effect of protecting the vascular endothelial cells, maintaining the normal permeability of the blood vessels, and reducing ischemic myocardial damage. Through a study on whether exogenous NADPH has an effect of treating ischemic myocardial injury, a new use of NADPH in treating myocardial injury and myocardial infarction were discovered. Specifically, the NADPH injected into the experimental mouse can pass the blood brain barrier and enter the cells. NADPH can maintain a normal permeability of the blood vessel after cerebral ischemia and reperfusion, which reduces the damage of blood brain barrier. NADPH can reduce the myocardial infarction range, and protect the heart from acute ischemic myocardial injury. The results above suggest that NADPH has a protection effect against ischemic myocardial injury, and that NADPH is an effective medicine for treating heart diseases, especially myocardial injury, myocardial infarction and cardiomyopathy.

Because NADPH is an endogenous antioxidant and also a substance for supplying energy, no toxic or side effect is found in the clinical application, which suggests the advantages of small dosage and high safety. In addition, NADPH can be orally taken, injected, and administrated through the nasal mucosa and the skin, which are all convenient. Moreover, because oxidative stress and energy metabolism disorder are common mechanisms which lead to ischemic injuries of other organs or tissues, the NADPH may be widely used in treating other diseases.

### BRIEF DESCRIPTION OF THE DRAWINGS

To make the contents of the present invention understood easily, the following detailed descriptions of the present invention are provided with reference to the accompanying drawings.

FIG. 1 shows the influence of exogenous NADPH on the survival rate of the primary endothelial cells HUVEC in low glucose and hypoxia environment.

FIG. 2 shows the influence of therapeutic administration of NADPH on the blood brain barrier related immune cells in the brain derived from mice with permanent cerebral ischemic stroke;

FIG. 3 shows the influence of preventive administration of NADPH on the blood brain barrier permeability in mice with cerebral ischemic reperfusion stroke;

FIG. 4 shows the effect of therapeutic administration of NADPH on the blood brain barrier damage of mice with permanent brain ischemic stroke; FIG. 4a shows blood brain barrier damage measurement result; FIG. 4b shows blood brain barrier permeability test result;

FIG. 5 shows the influence of therapeutic administration of NADPH on the myocardial injury in mice with myocardial ischemia reperfusion; FIG. 5a shows TTC staining result; FIG. 5b shows a comparison diagram of the weight percentage of the ischemic myocardial infarction zone in the total ischemic myocardial zone.

### DETAILED DESCRIPTION OF EMBODIMENTS

### Embodiment 1: Capsules Containing NADPH

The capsule of this embodiment comprises the following ingredients:

20 g of NADPH, 60 g of suspending agent microcrystalline cellulose; 0.04 g of preservative tert-butyl-4-hydroxy anisole; 2 g of lubricant magnesium stearate; with 200 g of filling agent lactose added.

The preparation method thereof comprises the following steps:

Weighing and mixing NADPH and medicinal auxiliary materials as listed in the prescription above; filtering the mixture for 3 times by using a 60-mesh sieve, and filling the filtered mixture into capsules.

Conventional auxiliary materials used in the preparation method comprise one or more selected from, but are not limited to, the following ingredients: filling agent, disintegrating agent, lubricant, adhesive, corrigent, suspending agent and preservative.

Specifically, the filling agent can be replaced with one or more of the following ingredients: pregelatinized starch, mannitol, chitin, microcrystalline cellulose and sucrose;

The disintegrating agent can be replaced with one or more of the following ingredients: starch, cross-linked povidone, sodium carboxymethyl cellulose and sodium carboxymethyl starch;

The lubricant can be replaced with one or more of the following ingredients: talcum powder, silicon dioxide and sodium dodecyl sulfate;

The suspending agent can be replaced with one or more of the following ingredients: polyvinylpyrrolidone, sucrose, agar and hydroxypropyl methyl cellulose;

The preservative can be replaced with one or more of the following ingredients: nipagin, benzoic acid and sodium benzoate, sorbic acid and sorbic acid salt;

The adhesive can be replaced with one or more of the following ingredients: polyvinylpyrrolidone, and hydroxypropyl methyl cellulose

The corrigent can be replaced with one or more of the following ingredients: sweetening agent and/or essence, wherein the sweetening agent includes one or more of the following: sodium saccharin, aspartame, sucrose and sodium cyclamate;

Of course, the conventional auxiliary materials include, but are not limited to, the ingredients listed above, a person skilled in the art can make adaptive selection and adjustment according to actual conditions.

A person skilled in the art can use any manner known in the art to administrate the medication provided in the present invention, including but not limited to external use, oral administration, sublingual, nasal, parenteral, local, subcutaneous, injection, transdermal or rectal administration. The pharmaceutical composition disclosed by the invention is preferably in oral dosage form and injection dosage form. The oral dosage form is selected from the following: oral liquid, capsule, effervescent tablet, oral film agent and spraying agent. The injection dosage form is selected from the following: powder-injection and liquid-injection dosage for muscle injection, subcutaneous injection or intravenous drip injection. The medication disclosed by the invention can be prepared into a corresponding dosage form by adopting methods known in the field.

### Experiment Examples

To prove the technical effect of the present invention, the following experiments are performed.

### Experiment 1

### The Protective Effect of Exogenous NADPH on Primary Endothelial Cells HUVEC

### (1) Experiment Materials:

The primary endothelial cells HUVEC were purchased from ATCC (the U.S.). Cryopreservation conditions: 2 mL cryopreservation tube, with 1.6 million cells per tube in 70% high-glucose DMEM, 20% domestic fetal bovine serum (FBS), and 10% DMSO.

### (2) Experimental Scheme

Culturing endothelial HUVEC cells: Culture condition: 37° C. (5% of CO₂ and 95% of air), saturated humidity, high-glucose DMEM culture medium supplemented with 100 U penicillin, 100 U streptomycin per liter and 10% domestic FBS; when cells grow to 80-90% confluence, carrying out cell passage by using trypsin-EDTA digestion. Passage frequency: passaging 5×10⁵ cells per bottle every 2-3 days. HUVEC cells in logarithmic growth phase are treated with trypsin-EDTA digestion solution to detach adherent cells. Cells are collected, counted and resuspended by using a medium containing 10% FBS to obtain a cell solution containing 5×10⁴ cells/mL. 100 μL of the cell solution is added to each well of a 96 well plate. The plate is cultured in 37° C. with 5 CO₂ for 24 hours. Prepare 10 mmol/L NADPH solution by using sterilized saline. After filtering sterilization, the NAPDH solution is added to the cell culture medium.

### MTT Assay for Testing Cell Viability:

### Cell Viability Assay:

Cells are inoculated in a 96 well plate. When cells are in logarithmic growth phase, adding NADPH with a final concentration of 1, 5, 10, 20 and 40 μM. The D-Hanks' solution only treatment is taken as the negative control. The plate is cultured in the incubator under the same condition for 48 hours. 4 hours before the end of culture, adding 10 μL MTT solution (5 mg/mL in D-Hanks' solution) to each well. At the end of the experiment, removing the supernatant and adding 150 μL DMSO. The OD value at 570 nm of each well is measured by using an ELISA reader. Average of 6 wells for each treatment is recorded.

the inhibition rate (IR %)=(1−OD of the test well/OD of the control well)×100% Calculating inhibition rate:

### (3) Experiment Result

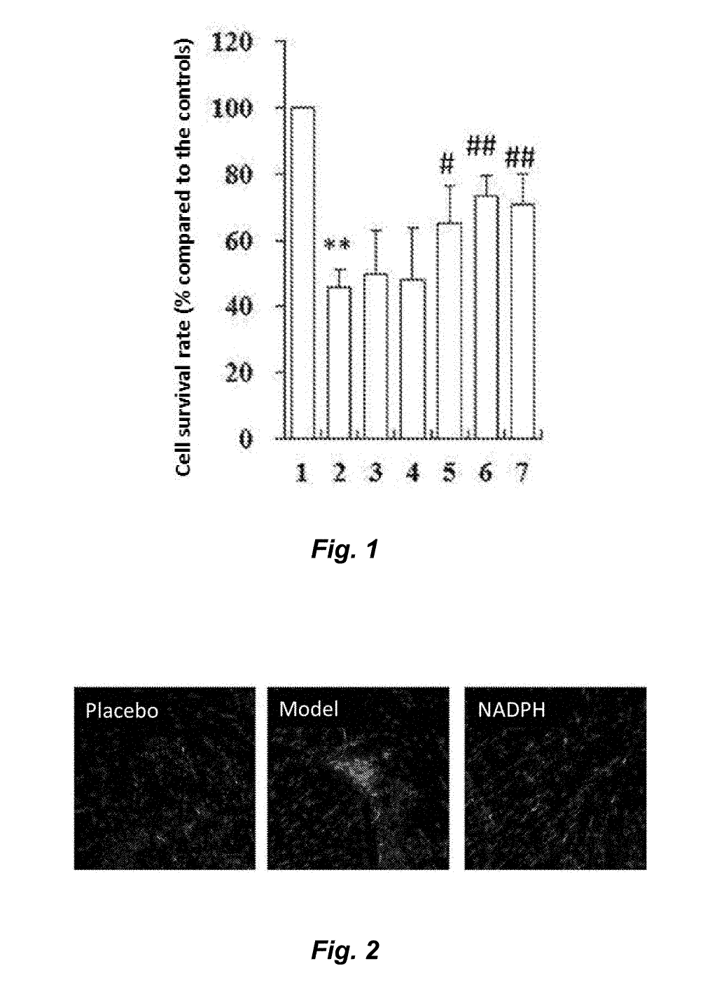

FIG. 1 shows the influence of the exogenous NADPH on the activity of the primary endothelial HUVEC cells in low glucose and hypoxia environment. As compared to the control group, treatment with NADPH at the concentration of 5, 10 and 20 μM for 48 hours significantly decreased the survival rate of HUVEC cells. The survival rate of cells treated with 5 μM NADPH for 48 hours is around 65.3% (p<0.05). The survival rate of cells treated with 10 μM NADPH for 48 hours is around 73.6% (p<0.01). The survival rate of cells treated with 20 μM NADPH for 48 hours is around 70.9% (p<0.01). 1) control group; 2) low glucose and hypoxia group; 3) low glucose and hypoxia group +NADPH 1 μM; 4) low glucose and hypoxia group +NADPH 5 μM; 5) low glucose and hypoxia group +NADPH 10 μM; 6) low glucose and hypoxia group +NADPH 20 μM; 7) low glucose and hypoxia group +NADPH 40 μM. * *, compared to the control group, p<0.01; #, compared to the low glucose hypoxia group, p<0.05; ##, compared to the low glucose hypoxia group, p<0.01. wherein ** indicates p<0.01, # indicates p<0.05, ## indicates p<0.01.

### Experiment 2

Preventive NADPH Administration to Alleviate the Blood Brain Barrier Related Immune Cell Response after Brain Injury

### (1) Experiment Materials

Clean-grade male ICR mice at the weight of 23-28 g were provided by the laboratory animal facility in Suzhou University, Laboratory animal producing license: XCYK(Su)2002-2008, laboratory animal using license: SYXK(Su)2002-0037. Male C57BL6 and Tg(Itgax-Venus)1Mnz mice at the weight of 22-27 g. Tg(Itgax-Venus)1Mnz mice with CD11c-eYFP positive are from MGI Inc., and are used to study the immune cell response of dendritic cells after brain injury. The breeding condition is as following: Room temperature 22° C., humidity 50-60%, well ventilation, artificial day and night shift (12 hours/12 hours), food and water available all the time. 2 days before the experiment, the mice are held in the environment for adaption. Dextran-Texas(Red) is from Invitrogen Inc.; NADPH is from Sigma reagent Co., Jiangsu; exogenous NADPH is obtained through artificial synthesis, semi-synthesis and biological extraction.

### (2) Experimental Scheme

Establishing permanent middle cerebral arterial embolism ischemia model. Tg(Itgax-Venus)1Mnz male mice at the weight of 22-27 g are used in this study. The experimental groups include: placebo operation only group, cerebrovascular injury model group, cerebrovascular injury +NADPH (2.5 mg/kg) treatment group, with 6 animals in each group. NADPH is diluted by using artificial cerebrospinal fluid and 2 μL of the NADPH solution is administrated through ventricular injection to the side cerebral ventricle (the cerebrovascular injury model group is injected with artificial cerebrospinal fluid). The mice are anesthetized by using chloral hydrate, fixed, incised in the center of the neck. The left common carotid artery, internal carotid artery and external carotid artery are separated. The near-heart end of the common carotid artery is ligated. A nylon wire with consistent diameter is inserted in front of the branch of the internal carotid artery and external carotid artery to block blood circulation for 24 hours. 22 hours after ischemia, Dextran-40 solution is injected through tail vein injection. After 2 hours of circulation, mice are anesthetized by using 10% chloral hydrate, and the chest is opened to expose the heart. A perfusion is performed on the left heart ventricle by using 10 ml 10 mmol/L PBS to remove the leftover Dextran-Texas in the brain tissue. NADPH is administrated 2 hours before preparing the permanent middle cerebral arterial embolism ischemia model. 24 hours after the establishment of the permanent middle cerebral arterial embolism ischemia model, the immune cell response related to the blood brain barrier is measured.

### (3) Experimental Method

Measurement of blood brain barrier related immune cell response: 22 hours after the brain microvascular injury, the mice are injected with Dextran-Texas via tail vein injection. 2 hours after the injection, mice are decapitated and the brain tissues are collected, which is followed by formalin perfusion, fixation and vibrating sectioning of the brain tissues, and immunohistochemical staining through the floating method is performed on the tissue slice collected in PBS solution. CD11c-eYFP antibody is used to mark positive dendritic cell, and DAPI is used to mark cell nucleus before seal. A confocal microscope is used to examine the distribution and expression of dendritic cell inflammatory response around the cerebrovascular in the striatum brain area.

### (4) Experiment Results

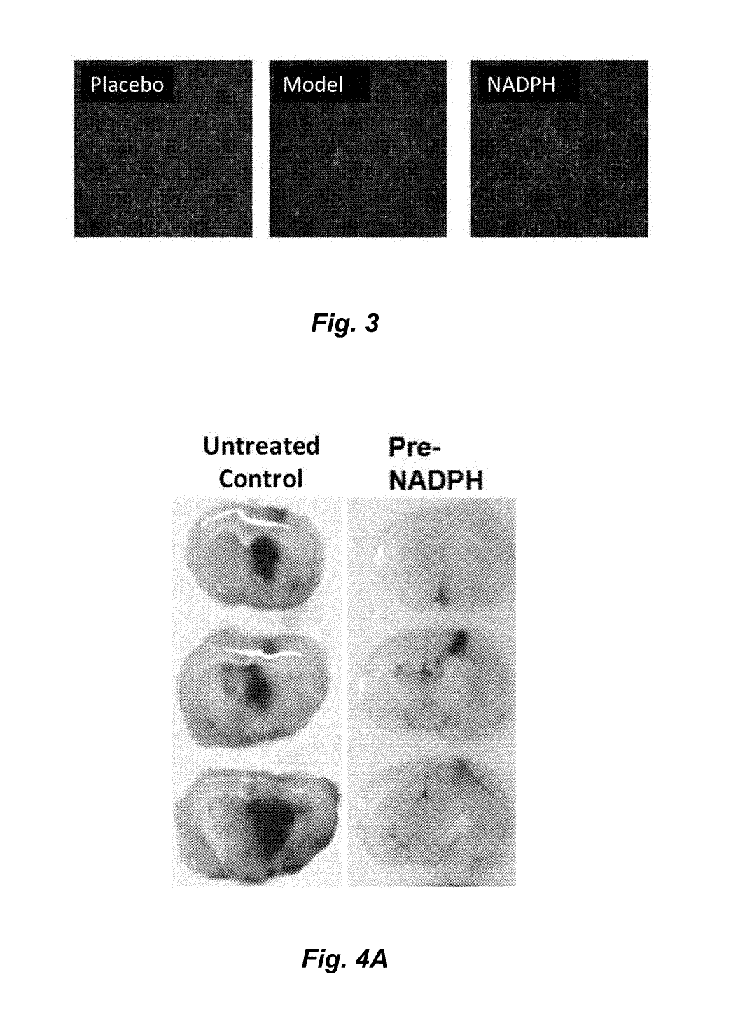

FIG. 2 shows the influence of preventive administration of NADPH on the local immune response in the mice with permanent brain ischemic stroke. In the mice treated with placebo operation only, CD11c-eYFP cells scatter in the brain regions, with small size and clear dendritic structure. 24 hours after the brain ischemia injury, the model group shows obviously responsive increasing of CD11c-eYFP cells in the striatum brain area, with increased size of the cells. Administration of NADPH (2.5 mg/kg) in the cerebral ventricle effectively reduces the responsiveness of CD11c-eYFP cells in the striatum brain area. This suggests that NADPH has a good protective effect on the blood brain barrier, which is related to the regulation of the local immune response.

### Experiment 3

The Preventive Administration of NADPH to Reduce the Injury of Blood Brain Barrier with Cerebral Ischemia Reperfusion

Experiment materials are the same as the Experiment 2

### (2) Experiment Process

### 1) Establishing Transient Middle Cerebral Arterial Occlusion Mouse Model

Normal ICR mice at the body weight of 23-28 g are randomly divided into two groups with 20 mice per group. One group is treated with saline (vehicle group), the other group is treated with NADPH (7.5 mg/kg). 1 week before the ischemia, NADPH is administrated via tail vein injection twice a day. An internal carotid artery suture method is adopted to modify the murine ischemic MCAO model. Mice are anesthetized using chloral hydrate (400 mg/kg) via intraperitoneal injection. We apply an ischemic model through the suture method, in which we separate the common carotid artery, external and internal carotid arteries, ligate the near-heart end of the external and common carotid arteries. The suture (Doccol Corporation, Redlands, USA) is inserted from the outside of the neck to the initial end of the anterior cerebral artery, which blocks the blood circulation in the middle cerebral artery. 2 hours after the blocking, the suture is removed to realize reperfusion. The placebo operation only group is treated in the same way as the ischemia group and the treatment group except that they don't get treated with the suture method. The whole operation is performed at 22-25° C. An automated heating pad is used to control the mice anal temperature at 37±0.5° C. After the operation, the mice are held in a feeding box with clean pad materials and plenty of food and water.

### 2) Establishing Permanent Middle Cerebral Arterial Occlusion Ischemia Mouse Model

Male C57BL/6 mice at the weight of 22-27 g are used in this study. The experimental groups include: placebo operation only group, cerebrovascular injury model group, cerebrovascular injury +NADPH (2.5 mg/kg) treatment group, with 6 animals in each group. NADPH is diluted by using artificial cerebrospinal fluid and 2 μL of the NADPH solution is administrated through ventricular injection to the side cerebral ventricle (the cerebrovascular injury model group is injected with artificial cerebrospinal fluid). The mice are anesthetized by using chloral hydrate, fixed, incised in the center of the neck. The left common carotid artery, internal carotid artery and external carotid artery are separated. The near-heart end of the common carotid artery is ligated. A nylon wire with consistent diameter is inserted in front of the branch of the internal carotid artery and external carotid artery to block blood circulation for 24 hours. 22 hours after ischemia, Dextran-40 solution is injected through tail vein injection. After 2 hours of circulation, mice are anesthetized by using 10% chloral hydrate, and the chest is opened to expose the heart. A perfusion is performed on the left heart ventricle by using 10 ml 10 mmol/L PBS to remove the leftover Dextran-Texas in the brain tissue. NADPH was administrated 2 hours before preparing the permanent middle cerebral arterial embolism ischemia model. 24 hours after the establishment of the permanent middle cerebral arterial embolism ischemia model, the immune response related to the blood brain barrier is measured.

### (3) Experimental Method

1) Blood brain barrier permeability test: 23 hours after the cerebral ischemia reperfusion, 2% Evan's blue (EB) in saline solution (4 ml/kg) is injected through the tail vein. 1 hour later, mice are decapitated, and the brain tissue is collected and weighed. The weighed brain tissue is put in 50% trichloroacetic acid solution, homogenized and centrifuged (10000 rpm, 20 min). The supernatant is removed and the tissue was diluted with ethanol at the ratio of 1:3. OD value at 620 nm is measured. The content of EB in the brain tissue is calculated.

2) Blood brain barrier injury test: 22 hours after the brain microvascular injury, Dextran-Texas is injected through tail vein. 2 hours later, mice are decapitated, the brain tissue is collected and fixed through formalin perfusion. Tissue slices are prepared through vibrating sectioning, and DAPI is used to mark cell nucleus before seal. A laser confocal microscope is used to examine the fluorescence intensity of Dextran-Texas in the solid brain tissue around the cerebrovascular area of the ischemic brain area.

3) Statistics and analysis: All data is expressed as the mean±standard error (Mean±SEM). The statistical analysis is performed by one-way ANOVA. p<0.05 represents that the statistical difference has significance.

### (4) Experiment Result

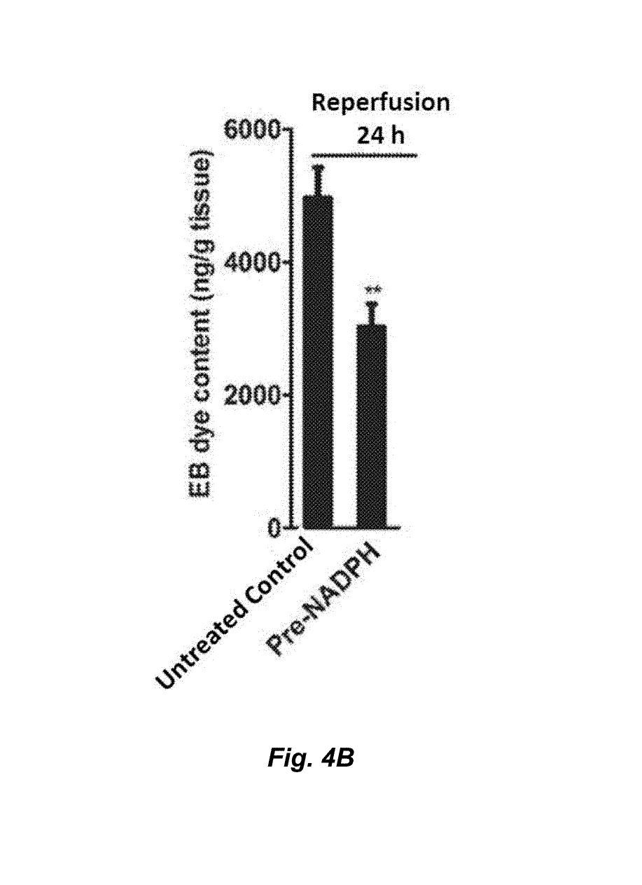

FIG. 3 shows the influence of preventive administration of NADPH on the permeability of the blood brain barrier in the mice with cerebral ischemia reperfusion stroke. As compared to the vehicle group, NADPH administration at one week before the stroke (twice per day through vein injection) significantly reduces the permeability of blood brain barrier in the mice with cerebral ischemic stroke (p<0.01). * * represents p<0.01.

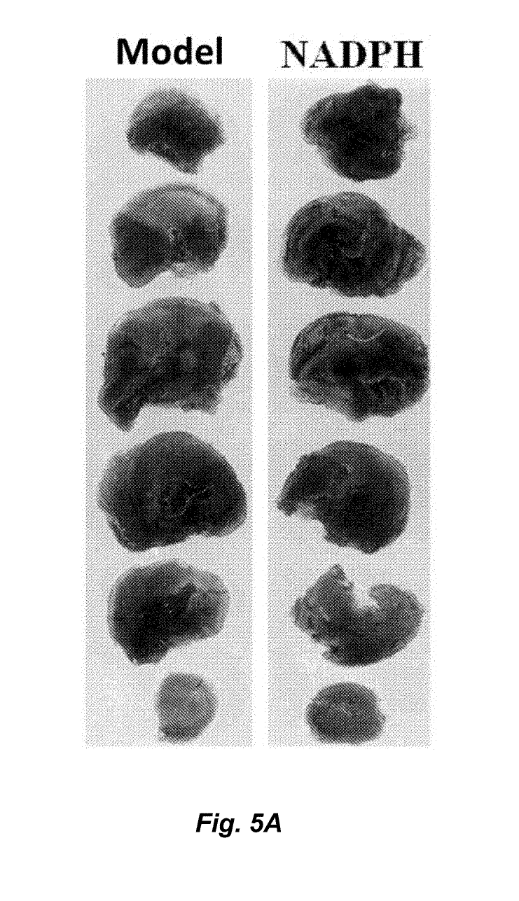

FIG. 4 shows the influence of preventive administration of NADPH on the permeability of the blood brain barrier in the mice with permanent brain ischemic stroke. FIG. 4a shows the results of blood brain barrier damage test. FIG. 4b shows the results of the blood brain barrier permeability test. 24 hours after the brain ischemia injury, as compared to the placebo operation only group, the model group shows significant red fluorescent dyes, namely the Dextran-Texas in the cerebral cortex and striatum area. The NADPH (2.5 mg/kg) treatment in the cerebral ventricle effectively reduced the ischemia induced Dextran-Texas leakage in the cerebral cortex and striatum area. This suggests that NADPH provides a protective effect for the blood brain barrier.

### Experiment 4

### Therapeutic Administration of NADPH to Reduce Myocardial Ischemia Injury

### (1) Experimental Materials

Clean-grade adult male SD rats at the body weight of 270-350 g are provided by the laboratorial animal facility in Suzhou University, License: XCYK(Su)2007-0035. Evans Blue (EB) is from Sigma Inc. Normal SD rats are randomly divided into 2 groups, including saline group (model group) and NADPH (7.5 mg/kg) dosage group, with 10 rats in each group. NADPH is immediately injected through tail vein during myocardial ischemia reperfusion.

### (2) Experiment Process

### Establishment of Rat In Vivo Myocardial Ischemia Reperfusion Injury Model

Adult male SD rats are injected with chloral hydrate via intraperitoneal injection for anesthesia. Catheters filled with heparin are placed in the right internal jugular vein and the internal carotid artery. The catheters are used for intravenous administration, the arterial blood gas analysis or arterial blood pressure monitoring. The trachea is incised and a tracheal catheter is inserted and connected to an ALC-V9 animal breathing machine which carries out positive pressure ventilation at the end of the breath. The inhaled oxygen concentration is 33%, the respiration frequency or the moisture volume is adjusted, and the breath is maintained at pH 7.35-7.45, PaCO225-40 mmHg, PaO290-150 mmHg. Through an intelligent thermostatic controller, the body temperature of the rat is maintained at 36-37° C. The left chest incision operation is carried out on the fifth rib interval, and the cardiac pericardium is opened. 6-0 damage-free suture line is used for ligating the left anterior descending coronary artery (LAD) at the lower edge of the left auricle. The end of the suture line is directed into a self-made ring tube and kept at balance for 30 min. The ring tube is clamped by a hemostatic forceps to block the LAD blood circulation. If the epicardium becomes pale, the electrocardiogram shows a transient arrhythmia, and the arch back of the ST section rises upwards, then the ischemia model is successful prepared. The ring tube is loosened for reperfusion, and the congestion of the epicardium is visible, which proves that the reperfusion is successful. Another 2 hours of reperfusion is carried out to detect the range of the myocardial infarction.

### (3) Experimental Method

Determining the myocardial infarction range: After the 30 min myocardial ischemia followed by the 2 hour reperfusion, the LAD is blocked again and 1 mL 5% EB is injected through the jugular vein, so that the normal area of left ventricle (LV) is stained to blue. Rapidly extracting the heart and separating the LV. The LV is cut into 5-6 pieces of tissue blocks with a thickness of 2 mm (FIG. 1-1-3) by using a rat heart sectioning device. The blue stained normal LV tissue and unstained LV ischemic tissue are separated. The TTC staining method is applied and the myocardial tissue is put into 0.5% TTC, followed by water bath incubation at 37° C. for 15 minutes. The tissue is fixed with 10% of formaldehyde overnight. Under the dissecting microscope, the LV tissues are divided into normal tissues, ischemic but not infarct tissues (red, risk zone), and ischemic infarct tissues (gray, infarct zone). In addition, we calculate the percentage of the weight of the ischemic infarct zone in the weight of the total ischemic myocardial zone. The range of the myocardial infarction is represented as the percentage of the weight of the ischemic infarct zone in the weight of the total ischemic myocardial zone.

### (4) Experiment Result

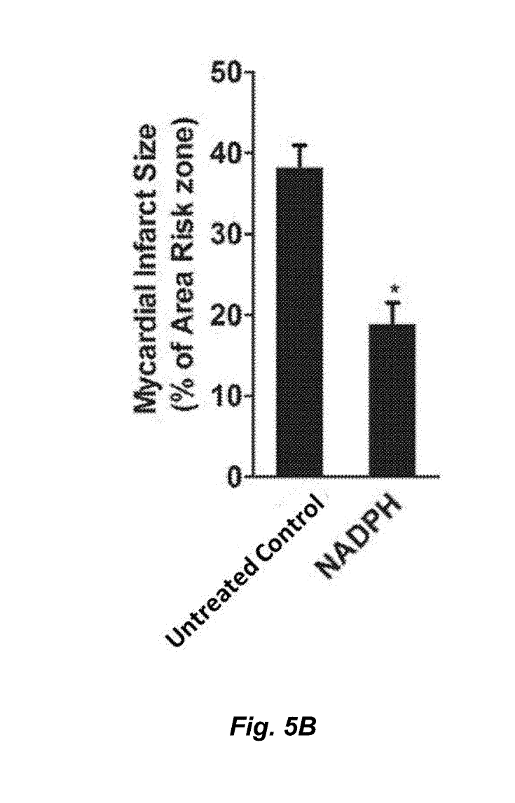

FIG. 5 shows the influence of the therapeutic administration of NADPH on myocardial ischemia infarction. FIG. 5a shows TTC staining result; FIG. 5b shows a comparison diagram of the weight percentage of the ischemic myocardial infarction zone in the total ischemic myocardial zone. TCC staining result shows that: as compared to the vehicle group (the control group), administration of NADPH through tail vein injection at the 0 hour of reperfusion significantly reduces the range of myocardial infarction in the rats (p<0.05). This suggests that NADPH reduces the myocardial ischemic injury. The blue part represents the non-ischemic area, the red part represents the ischemic area, and the gray part represents the infarction zone. Wherein * represents p<0.05.

Apparently, the above-described embodiments are merely examples for clearly illustrating the present invention, and are not intended to limit the implementation ways of the present invention. For a person skilled in the art, various changes and modifications in other different forms can be made on the basis of the description above. It is unnecessary and impossible to exhaustively list all the implementation ways herein. Any obvious changes or modifications based on these embodiments are still within the protection scope of the present invention.

1. The use of NADPH in preparation of a medicine for treating a heart disease.

2. The use according to claim 1, wherein the heart disease is selected from the group of myocardial injury, myocardial infarction and cardiomyopathy.

3. The use according to claim 2, wherein the cardiomyopathy is a hypertrophic cardiomyopathy.

4. The use according to claim 1, wherein the medicine comprises a pharmaceutically effective amount of NADPH and a pharmaceutically acceptable carrier.

5. A medicine for treating heart diseases, wherein NADPH is the active ingredient of the medicine, and the medicine is prepared by adding conventional auxiliary ingredients to NADPH so as to produce a clinically acceptable mixture, capsule, tablet, medicinal film agent or spraying agent according to a conventional process.

6. A medicine for treating myocardial injury, myocardial infarction or cardiomyopathy, wherein NADPH is the active ingredient of the medicine, and the medicine is prepared by adding conventional auxiliary ingredients to NADPH to produce a clinically acceptable mixture, capsule, tablet, medicinal film agent or spraying agent according to a conventional process.

7. The use according to claim 2, wherein the medicine comprises a pharmaceutically effective amount of NADPH and a pharmaceutically acceptable carrier.

8. The use according to claim 3, wherein the medicine comprises a pharmaceutically effective amount of NADPH and a pharmaceutically acceptable carrier.
